(19) United States
(12) Patent Application Publication
(10) Pub. No.: US 2017/0101616 A1
(43) Pub. Date: Apr. 13, 2017

(54) SONICATED BIOLOGICAL HYDROGEN REACTOR

(71) Applicant: THE UNIVERSITY OF WESTERN ONTARIO, London (CA)

(72) Inventors: Elsayed Elrefaey ELBESHBISHY, London (CA); George F. NAKHLA, Woodbridge (CA)

(21) Appl. No.: 15/055,283

(22) Filed: Feb. 26, 2016

Related U.S. Application Data
(63) Continuation of application No. 13/308,085, filed on Nov. 30, 2011, now abandoned.

(51) Int. Cl. C12M 1/42; C12P 3/00; C12M 1/107; C02F 3/00; C12M 3/06; C02F 3/28; C02F 1/36; C12P 5/02; C12M 1/34

(52) U.S. Cl. CPC C12M 35/04; C12P 5/023; C12P 3/00; C12M 21/04; C12M 41/26; C12M 27/16; C12M 41/12; C02F 3/28; C02F 1/36; C02F 3/006; C02F 2209/001; C02F 2209/003; C02F 2209/02; C02F 2209/06; C02F 2209/44

(57) ABSTRACT

A method and system for hydrogen production from organic material such as waste. The system includes a bioreactor for continuous anerobic fermentation to produce hydrogen in which a mixture containing a microorganism and organic material is sonicated. The system optionally includes a biomethanator connected in-line with the bioreactor.

### FIELD OF THE INVENTION

The present invention relates to a method of anaerobically digesting organic material and an apparatus therefor. Anaerobic digestion includes sonication of a fermentation mixture to produce hydrogen.

### BACKGROUND OF THE INVENTION

Anaerobic digestion processes that can convert the organic wastes to produce useful products such as hydrogen and methane are known. In a typical process, the waste is first subjected to hydrolysis or solubilization where e.g., biosolids and/or particulate organic substrates are broken down allowing the organic matter to be more readily transformed in subsequent microbial digestion steps. [1] Generally speaking, acidification can follow with subsequent methanogenesis. Organic matter can be converted to hydrogen and volatile fatty acids by hydrogen-producing bacteria during acidification, with subsequent microbial conversion of hydrogen and volatile fatty acids to methane by methanogens. Generally speaking, the rate-limiting step of anaerobic digestion of organic waste is often the first step of hydrolysis or solubilisation, so anaerobic digestion could be improved by enhancement of hydrolysis. Thus, pretreatment of the organic material to be digested is often conducted in order to achieve the release of intracellular polymers, and solubilization of particulate substrates e.g., lignocellulosic material to accelerate the subsequent conversion of the organics to biogas by microbes during anaerobic digestion[2]. Various pretreatment methods such as thermal, chemical, physical, and biological have been studied over the years [3].

Hydrogen, as an energy carrier, offers numerous advantages over other conventional energy carriers. An advantage of hydrogen as an energy source is the absence of polluting emissions since the utilization of hydrogen, either via combustion or via fuel cells, produces water [4]. At present, hydrogen is produced primarily from fossil fuels, biomass, and water using chemical or biological processes. Anaerobic (or dark) fermentation and photosynthetic degradation are the two most widely studied biohydrogen production techniques. [1] Anaerobic fermentation is promising for sustainable hydrogen and methane production since organic matter, including waste products, can be used as a feedstock for the process. [5] However, the rate of biological hydrogen (H₂) production is low and the technology needs further development. [6]

In the context of anaerobic digestion, hydrogen partial pressure and the resulting H₂ concentration in the liquid phase are key factors affecting fermentative H₂ production [3]. Generally, high H₂ partial pressure has a negative effect on H₂ production by decreasing the activity of the enzyme hydrogenase and making the H₂ production reaction thermodynamically unfavourable [7]. Various techniques have been used to remove metabolic gases (H₂, CO₂) from the liquid phase. [8] Gas sparging has been a common method used to decrease the concentrations of dissolved gases in fermentative H₂-producing bioreactors. Other techniques to decrease concentrations of dissolved gases include increased stirring [9], decreasing the reactor headspace pressure i.e. applying a vacuum [10], and using an immersed membrane to directly remove dissolved gases [10]. The disadvantage of gas sparging is that the sparging gas should be free of CO₂ because of its ability to inhibit hydrogenase [7]. In addition, too much sparger gas dilutes the H₂ content in the headspace and creates problems in the separation and utilization of the biogas [11].

Ultrasonication is a means for causing a localised pressure in a liquid to drop to below the evaporating pressure in the aqueous phase, which results formation of micro-bubbles or cavitation bubbles. [12] During cavitation, micro-bubbles form at various nucleation sites in the fluid and grow during the rarefaction phase of the sound wave. [13]. In the subsequent compression phase, the bubbles implode and the collapsing bubbles release a violent shock wave that propagates through the medium [14].

Ultrasonication can disrupt biosolids flocs and bacterial cells, releasing intracellular components, to improve the rate of anaerobic degradation due to the solubilisation of the particulate matter. This can decrease the required solids retention time (SRT) and improve the overall performance of anaerobic digestion [15]. The use of ultrasonication in the pretreatment of waste activated sludge (WAS) has also been found to improve the operational reliability of anaerobic digesters, decrease odor generation and clogging problems, and enhance sludge dewatering. [16] Ultrasonication can enhance hydrogen production when applied inside the bioreactor. The mechanisms for enhancement of hydrogen production by ultrasonication inside the bioreactor include: (1) decreasing the dissolved hydrogen concentration; (2) enhancement of the mass transfer; (3) increasing the microorganisms' growth rate; and/or (4) solubilization. Decreasing the dissolved H₂ concentration is known to increase the H₂ production via one of two possible scenarios: (i) increasing the H₂ production, or (ii) decreasing the H₂ consumption. H₂ generation is mediated by hydrogenase using electrons from ferreodoxin (Fd) to reduce protons.

A description of the use of sonication in anaerobic digestion that produces methane as an end product is given by Yoshitani et al. in United States Patent Publication No. 2006/0172405, published Aug. 3, 2006. Yoshitani et al. stated that in a particular embodiment hydraulic retention time (HRT) could be reduced from about 20 days to about 5 days through the use of sonication.

There have been studies investigating the effects of ultrasonication on biological hydrogen production. Three studies looked at ultrasonication of sewage sludge as a substrate [17-19], and the other three applied ultrasonication to the seed biomass [20-22]. Guo et al. [20] studied the impact of ultrasonic pretreatment on hydrogen production from boiled anaerobically digested sludge at 90° C. for 15 min with sucrose as substrate. In another study, More and Ghangrekar [21] evaluated the effect of ultrasonication pre-treatment on mixed anaerobic sludge to inoculate the microbial fuel cells, and reported that the ultrasonication pre-treatment of 5 min affected a maximum power density 2.5 times higher than the untreated sludge. Moreover, in a previous study involving the inventors named herein, using batches, the effect of ultrasonication on eliminating methanogenesis and therefore enhancing the bio-hydrogen production was examined. [22] The optimized sonication energy for hydrogen production using anaerobically digested sludge was 79 kJ/g TS (total solids) and the hydrogen yield increased by 45% compared with the untreated sludge.

### SUMMARY OF THE INVENTION

The inventors have established the feasibility of continuously fermenting a mixture of a hydrogen-producing anaerobic microorganism and organic material in a bioreactor to produce hydrogen. The continuous fermentation process can be operated reliably for an extended period without interruption.

In one aspect, the invention is a method of anaerobically digesting organic material. The method includes steps of:
(a) continuously fermenting a mixture of a hydrogen-producing anaerobic microorganism and a portion of the organic material in a bioreactor to produce hydrogen;
(b) drawing gaseous hydrogen produced in step (a) from a headspace above the mixture in the bioreactor; and
(c) feeding another portion of the material having an organic load (00) into the bioreactor to supplement the organic load of the material in the bioreactor; and
(d) removing a portion of digested material from the bioreactor.

It is possible to obtain an average rate of production of hydrogen in step (a) that is at least 20% of the average organic loading rate (OLR). Here, hydrogen production rate is measured as unit volume of H₂ produced per unit volume of the bioreactor per unit time. Typically, this is measured in L/L_{bioreactor}·d. The organic loading rate is the weight of COD (chemical oxygen demand) per unit bioreactor volume per unit time. Typically, this is measured in g COD/L_{bioreactor}·d. Step (a) of the method includes intermittently sonicating the mixture in the bioreactor up to 90% of the time. Steps (c) and step (d) are carried out so as to obtain a hydraulic residence time (HRT) of the organic material in the bioreactor of between 2 and 48 hours and the stated hydrogen production rate. Steps (c) and (d) can be operated continuously, as appropriate, or one or the other or both can be operated repeatedly, to control the hydraulic residence time of the material within the bioreactor. It might be suitable, for example, for input of the material in step (c) to be operated continuously if the feedstock organic material of step (c) is a liquid.

The invention thus includes a method in which hydrogen is produced continuously through fermentation. There will, of course, be variation in the rate of production of hydrogen over time. As described in detail below, in the steady-state, less than 10% variation in biogas quantity was observed over many days. The absolute rate of production of hydrogen depends upon the nature of the feedstock, the rate of input of feedstock, the anaerobic microorganisms present in the bioreactor, temperature, etc.

The method of the invention would typically be run over a period of time in which hydrogen is produced continuously i.e., where fermentation proceeds, even though the hydrogen production rate can vary over time due to changes in feedstock, etc. The process can be run with multiple turnovers, a turnover period being the HRT. The studies described herein were run in the steady-state for well over two months with an HRT of about 12 hours. It is thus possible to operate a process of the invention over multiple turnovers (e.g., at least 2, 4, 6, 8, 10, 20, 50, 100, 500, or more).

According to the invention, over a period of time, the average rate of production of hydrogen, in the units specified above, is at least 20% of the average organic loading rate, in the units specified. It is possible to obtain higher performance than this over a period of time. For example, an average rate of production of hydrogen that is at least 25%, 30%, 35%, 40% or more of the average organic loading rate is possible.

According to the invention, over a period of time, the average rate of production of hydrogen is at least 20% of the average organic loading rate. It is possible to obtain higher performance than this over a period of time. For example, an average rate of production of hydrogen that is at least 25%, 30%, 35%, 40% or more of the average organic loading rate is possible.

The hydrogen-producing microorganism present can be bacteria, archaea, protozoa, fungi, one or more strains of Acetobacter sp., Gluconobacter sp., or Clostridium sp., mesophilic bacteria, thermophilic bacteria, etc., as described further below.

The temperature in the interior of the bioreactor is controlled, if desired or needed, to operate at a temperature conducive to fermentive hydrogen formation, between 30° C. and 45° C., or between 35° C. and 40° C., or between 50° C. and 65° C., or between 55° C. and 60° C., etc.

Metabolic processes of microorganisms can vary in response to environmental factors, such as pH. It may thus be necessary or desirable to maintain the pH within the bioreactor to between, for example, 4.5 and 6.5, or between 5 and 6.

According to an aspect of the invention, the method includes monitoring the pH of the fermentation mixture, and adjusting the pH by adding to the bioreactor, soda ash, sodium bicarbonate, sodium hydroxide, calcium hydroxide, magnesium hydroxide, nitric acid, hydrochloric acid, or a combination of any of the preceding.

The HRT can be as low as 2 hours and as high as 48 hours, but could also be between 2 hours and 18 hours, between 2 hours and 12 hours, between 3 hours and 10 hours, or between 4 and 6 hours.

The bioreactor mixture is intermittently sonicated up to 90% of the time the fermentation process is underway during continuous operation. The proportion of time could also be between 1% and 80%, 1% and 70%, 5% and 60%, 10% and 60%, 15% and 60%. 20% and 60%, 25% and 60%, 30% and 60%, 30% and 50%, or between 30% and 40% of the time.

In the studies carried out herein, the sonicator horn was located in the bioreactor, and was in direct contact with the fermentation mixture so that the sonication energy directly impinges upon the mixture.

In another aspect of the invention, agitating the mixture in the bioreactor is included. The mixture can agitated by mechanically agitating the bioreactor, stirring the mixture, gas mixing the mixture, jet mixing the mixture, etc.

The sonication frequency would usually be in the range of 1 kHz to 20000 kHz, but in other aspects, the invention includes use of sonication frequency in the range of 20 to 10,000 kHz, or 20 to 1,000 kHz, or 20 to 500 kHz, or 20 to 500 kHz, or 20 to 100 kHz.

The organic material includes, but is not limited to sewage sludge, an organic fraction of municipal solid waste, industrial waste, food processing waste, agricultural waste, manure, residuals of bioethanol production, dedicated energy crops; alcohol, a ketone, an aldehyde, a volatile fatty acid, an ester, an ether, or a combination of any of the preceding; a carboxylic acid; a carbohydrate, a protein, a lipid, a nucleic acid; polysaccharide; monosaccharide; cellulose, including combinations of the foregoing, or mixtures of material that include any of the foregoing.

The bioreactor can include a temperature controller to control the temperature of the mixture in the bioreactor.

The method can include adding one or more nutrients to the bioreactor to promote the growth of the microorganisms or other metabolic processes, to enhance hydrogen production. Typical nutrients are nitrogen containing compounds, phosphorous containing compounds, iron, manganese, magnesium, calcium, cobalt, zinc, nickel, and/or copper.

In other aspects of the invention, the bioreactor is operated continuously for at least a day, 2 days, 3 day, 4 days, 5 days, 6 days, 7 days, 14 days, 3 weeks, or 4, 5, 6, 7, 8, 9, 10, 11, 12, 13, 14, 15, 16 17, 20, 25, 30, 35, 40, 45, or at least 50 weeks.

In another aspect of the invention, hydrogen-producing anaerobic microorganisms are supplied into the bioreactor at least once. This may be during a start-up period, or may be to supplement those organisms already contained in the bioreactor. In the studies described below, the microorganisms freely intermixed with the organic material in the bioreactor i.e., were unanchored (not immobilized).

In another aspect of the invention, hydrogen-producing anaerobic microorganisms are supplied into the bioreactor as a component of sludge at least once. This may be during a start-up period, or may be to supplement those organisms already contained in the bioreactor. In the studies described below, the microorganisms freely intermixed with the organic material in the bioreactor i.e., were unanchored (not immobilized).

A bioreactor of the invention would, according to certain aspects of the invention, be a vessel having outlet to provide egress of digested (or partially digested) material from the bioreactor. The outlet can be, for example, connected to a conduit connected to a biomethanator downstream of the bioreactor, for delivery of the material from the bioreactor to the biomethanator.

A biomethanator can be a single or multi-stage continuously stirred tank reactor (CSTR), up-flow anaerobic sludge blanket reactor (UASB) in which a waste stream flows upwards through an anaerobic compacted bed of granular sludge, an expanded bed granular sludge blanket (EGSB) in which waste flows upwards through an anaerobic expanded granular sludge, a down-flow or up-flow anaerobic granular media reactor, an anaerobic baffled tank reactor (ABR), an anaerobic migrating blanket reactor (AMBR), or an anaerobic fluidized bed (AFB) bioreactor.

Products of fermentation in the bioreactor, in addition to hydrogen and other molecules, can include in various amounts, volatile fatty acids, and/or alcohols. These can act as feedstock for biomethanogenesis.

A conduit of the bioreactor can include an in-line chamber located between the bioreactor and the biomethanator. The chamber can be for e.g., adjusting the pH of material from the bioreactor prior to feeding the material into the biomethanator.

A bioreactor of the invention can include an outlet connected to a downstream processor into which digested material is fed through the outlet to the processor. Solids and liquids can be separated in the processor. Such separation can be, for example, by gravity settling, centrifugation, belt separation, frame pressing, filtration and/or by membrane separation.

### BRIEF DESCRIPTION OF THE DRAWINGS

A detailed description of aspects of the invention is provided below, with reference to accompany drawings, in which:

FIG. 1 is a schematic of an apparatus for implementing the invention, and illustrates the bioreactor used to obtain the results described herein. The schematic includes a particular embodiment of a bioreactor, the includes an optional biomethanator a treatment tank situated in-line between the two reactors and downstream of the bioreactor;

FIG. 2 shows hydrogen production rates (L H₂ produced/L·d volume of bioreactor) as a function of time (days). The bioreactor was operated in batch mode for the first 24 hours, and continuous mode thereafter. The organic loading rate (OLR) was 21.4 g COD/L·d for phase 1 and 32.1 g COD/L˜D for phase 2. The filled points (upper) are for the sonicated and stirred bioreactor (SBHR) and the hollow points (lower) are for the stirred (unsonicated) bioreactor (CSTR). The hydraulic retention time (HRT) was 12 hours;

FIG. 3 shows hydrogen yields (mol H₂/mol glucose) as a function of time (days) for the SBHR (filled points) and the CSTR (hollow points) in phases 1 and 2; and

FIG. 4 shows the yield of biomass (g VSS/L) as a function of cumulative SCOD (g/L) for Phase 1 (SBHR (), CSTR(∘)) and Phase 2 (SBHR (open triangle), CSTR (▴));

FIG. 5 is DGGE profiles of the 16S rDNA gene fragment at each treatment condition, obtained from total DNA from samples extracted from the CSTR and SBHR, followed by PCR; and

FIG. 6 shows the correlation between food to microorganisms (F/M) ratio and hydrogen yield, comparing values obtained using the methods of this invention to literature values: CSTR, literature (∘); CSTR, gas-sparging literature (); SBHR (▴); and CSTR (open triangle).

### DETAILED DESCRIPTION OF THE INVENTION

Embodiments of the present invention are disclosed herein. However, the disclosed embodiments are exemplary, and it is to be understood that the invention may be embodied in many various and alternative forms. The Figures are intended to aid in the understanding of the invention and may not be to scale, with some features exaggerated or minimized. Therefore, specific structural and functional details disclosed herein are not to be interpreted as limiting but as a basis for the claims and as a representative basis for teaching one skilled in the art to variously employ the present invention.

The term “about”, when used in conjunction with ranges of dimensions, temperatures or other physical properties or characteristics is used as it would be by a skilled person in a similar context. Typically, the term is meant to cover slight variations that may exist in the upper and lower limits of the ranges of dimensions so as to not exclude embodiments where on average most of the dimensions are satisfied but where statistically dimensions may exist outside this region.

As used herein, the term “organic material” refers to any material containing carbon that may be anaerobically digested, as by hydrogen-producing anaerobic microorganisms, to produce hydrogen. Organic material of the invention will often be “organic waste” such as plant material, municipal waste such as sewage sludge and solid waste, industrial waste, such as food processing waste, agricultural materials, such as manure, residues, and dedicated energy crops, wastes that include carbon and hydrogen such as, but are not limited to, alcohols, ketones, aldehydes, volatile fatty acids, esters, carboxylic acids, ethers, carbohydrates. proteins, lipids. polysaccharides, monosaccharide, cellulose, and nucleic acids. The material can include plant waste (e.g., agricultural waste or crop waste), animal material, food waste, industrial waste, and organic waste products and/or residues thereof. The waste can contain cellulose or hemicellulose, mixtures, combinations, derivatives, or residuals thereof. Cellulose is present in plant cell walls and is a significant component of plant matter, including cotton. Cellulose is comprised of glucose. Cellulose can be difficult to break down because of its crystalline structure. Hemicellulose is composed of many different sugar monomers, and is usually easily hydrolyzed. Sources for cellulose and hemicellulose include, but are not limited to, the plant materials provided above (e.g., corn stover, wheat straw, soybeans, hay, cotton, grain sorghum, barley, oats, rice, rye, forest residue, mill residue, agricultural waste and residue thereof, urban wood waste and residue thereof, and dedicated energy crops). Organic material of the invention can be forest residue, mill residue, agricultural waste and residue thereof, urban wood waste and residue thereof, and dedicated energy crops. Forest residue may include, for example, logging residue; rough, rotten, or salvable dead wood; excess saplings; and small pole trees. Mill residue may include, for example, bark; coarse residues (e.g., chunks and slabs); and fine residues (e.g., shavings and sawdust). Agricultural waste and residue may include, for example, stalks and residue from e.g., corn (e.g. corn stover), wheat (e.g. wheat straw), soybeans, hay, cotton, grain sorghum, barley, oats, rice, and rye. Urban wood waste and residue may include, for example, yard trimmings, site clearing wastes, pallets, wood packaging, and other miscellaneous commercial and household wood wastes. Dedicated energy crops may include, for example, short rotation woody crops such as hybrid poplar and hybrid willow, herbaceous crops such as switchgrass, and woody non-stem residue. Exemplary feedstock includes, for example, corn stover. The organic material can include food waste, food processing waste, and animal waste and waste products (e.g., livestock manure). Lipid-rich waste such as glycerol and animal fat may also be used as a biomass feedstock. Organic waste such as the organic fraction of municipal solid waste, construction waste, and demolition waste may also be used as biomass feedstock. In certain embodiments, a carbohydrate-rich source or carbohydrate-rich mixture (e.g., combining two, three, four or more biomass feedstock sources) may be used for hydrogen production. Organic material of the invention can also include pentose products (e.g., xylose, arabinose, mixture of polymers that contain xylose, arabinose, etc), hexose products (e.g., mannose, glucose, galactose, mixture of polymers that contain mannose, glucose, galactose, etc), volatile products ((e.g., volatile fatty acids (as acetic acid, butyric acid, and propionic acid, etc), sugar acids (as gluconic acid, uronic acid, glucouronic acid, etc), organic solvent (as ethanol, methanol, propanol, etc), and volatile organic compounds (as aldehyde, ketone, hydrocarbon, etc)), and inhibiting compounds (e.g., furfural and soluble lignin compounds) may be used for methane production. Organic material of the invention can be waste products from a bioethanol production process, in particular distiller's grain solids (DGS) and dry distiller's grain solids (DDGS). The material to be digested can include liquids and/or solids, and can include material that is not digestable, but that passes through the bioreactor undigested, such as inorganic sediment. Organic material treated by methods of the invention can be non-sterile, and the material need not be required to be pretreated in a manner that destroys or renders unfunctional microorganisms that are typically found in such materials e.g., methanogens.

As used herein, a “bioreactor” refers to a vessel that can be anaerobically sealed during its operation to permit microorganisms within the vessel to digest through a fermentation process the organic material. The bioreactor can be set up to agitate its contents. This can be achieved by mechanically agitating the bioreactor itself, the use of an internal stirring mechanism, gas-mixing, or other suitable means of mixing the microorganisms and/or organic material.

A “hydrogen-producing microorganism” is a microorganism that can ferment organic material under anaerobic conditions to produce hydrogen (H₂). Other products of fermentation can include carbon dioxide, a variety of organic acids and alcohols, etc. The microorganisms include bacteria, archaea, protozoa, fungi, and other microorganisms which can digest the organic material to produce hydrogen. Specific examples of such microorganisms are Acetobacter sp., Gluconobacter sp., Enterobacter cloacae, Bacillus circulans, Citrobacter freundii. and Clostridium sp.

Hydrogen production of the invention is “continuous” i.e., once hydrogen production is suitably established, fermentation to produce hydrogen continues uninterrupted, although the production may vary over time, for example, depending upon variation in feedstock influent flow and characteristics, etc. The organic material is typically fed into the vessel continuously, and the product hydrogen and digested material are also withdrawn continuously, and this is done without stopping the digestion process within the vessel. This is not to say, that the method could not be operated such that feedstock is fed intermittently into the digestion vessel, or that hydrogen is withdrawn intermittently, or that digested material is intermittently removed. The nature and timing of these steps are adapted to the nature of the feedstock, available apparatus elements, process controls, etc.

“Sonication” refers to the application of sound waves (acoustic energy) transmitted through a liquid medium (manure, water, oil, etc.). Ultrasonication, used interchangeably herein with the term sonication, causes a localised pressure drop to below the evaporating pressure in the aqueous phase, resulting in the formation of micro-bubbles or cavitation bubbles. During cavitation, micro-bubbles form at various nucleation sites in the fluid and grow during the rarefaction phase of the sound wave. Subsequently, in the compression phase, the bubbles implode and the collapsing bubbles release a violent shock wave that propagates through the medium. The sonication energy may be applied to the organic waste in the bioreactor, and if present in the biomethanator; the frequency applied to the organic waste in the bioreactor or in the biomethanator can be within any range.

The sonication energy depends on the size, shape of the vessel (bioreactor or methanator), and characteristics of the organic material being digested. The sonication energy source comprises a power source connected to a wave-generator connected to a converter (transducer) connected to a booster that is connected to a horn (sonotrode), plate, or any other kind of device delivering the sonication energy to the SBHR and/or to the biomethanator. A converter basically converts electrical energy into ultrasound energy (vibration). The booster is a mechanical amplifier that helps to increase the amplitude generated by the converter. The horn is a specially designed tool that delivers the ultrasonic energy to the sludge.

A “biomethanator” is one of any of the common designs used for the anaerobic conversion of organic wastes to methane and carbon dioxide. A biomethanator can be, but is not limited to, a single or multi-stage continuously stirred tank reactor (CSTR), an up-flow anaerobic sludge blanket (UASB) reactor where in the waste stream flows upwards through an anaerobic compacted bed of granular sludge, an expanded bed granular sludge blanket (EGSB) reactor in which waste flows upwards through an anaerobic expanded granular sludge, or a down-flow or up-flow anaerobic granular media reactor, an anaerobic baffled tank reactor (ABR), an anaerobic migrating blanket reactor (AMBR), or an anaerobic fluidized bed (AFB) bioreactor.

A step of the method of the invention involves “drawing” gaseous hydrogen from the bioreactor. This may be done with the aid of a vacuum, or if circumstances suit, a valve through which hydrogen is released from the vessel at a particular pressure, etc.

Another step of the invention involves removing a portion of the organic material that has been digested. This step can be carried out in any conventional way. A bioreactor for digesting material containing solids that remain throughout the process will be set up for removal of such remaining material or grits, and preferably this will be done as digestion proceeds so as not to interrupt hydrogen production.

The residence time of material in the bioreactor or digester vessel of the invention is a temporal gauge of the movement of organic material being processed from the point it is fed into the bioreactor to the point at which it exits or is removed from the bioreactor. For the purposes of this invention, “Hydraulic retention time” (HRT) is the volume of the bioreactor divided by the influent flowrate: HRT=(Volume of bioreactor)/(influent flowrate). Of course the inflow rate of the organic material (influent) and outflow rate of digested material generally match each other over time so as to maintain a relatively constant average volume of material within the vessel. This is not to say that the volume of material within the vessel could not be adjusted from time to time to suit particular circumstances.

“Organic loading rate” (OLR) is a measure of the amount of the microbially digestible material contained in the organic material entering a bioreactor of the invention. “Organic load” (OR) is defined in terms of chemical oxygen demand (COD), and so OLR follows from this in practice as being defined as the rate of input of the COD of the organic material into the bioreactor.

“Organic loading rate” (OLR) is a measure of the amount of the organic material entering the bioreactor of the invention per unit time per unit bioreactor volume. “Organic content” (OC) is defined in terms of chemical oxygen demand (COD), and so OLR follows from this in practice as being defined as the rate of input of the COD of the organic material into the bioreactor per unit bioreactor volume. OLR can thus be measured e.g., in units of CODₘₐₛₛ/bioreactor volume·time i.e. g COD/L_{bioreactor}·d, kg COD/m³_{bioreactor}·d, etc.

“Chemical oxygen demand” (COD) is a known measure of the amount of organic content of the organic material feedstock of the invention. Here, a HACH Odyssey DR/2500 kit was used, but other methods are known to the skilled person.

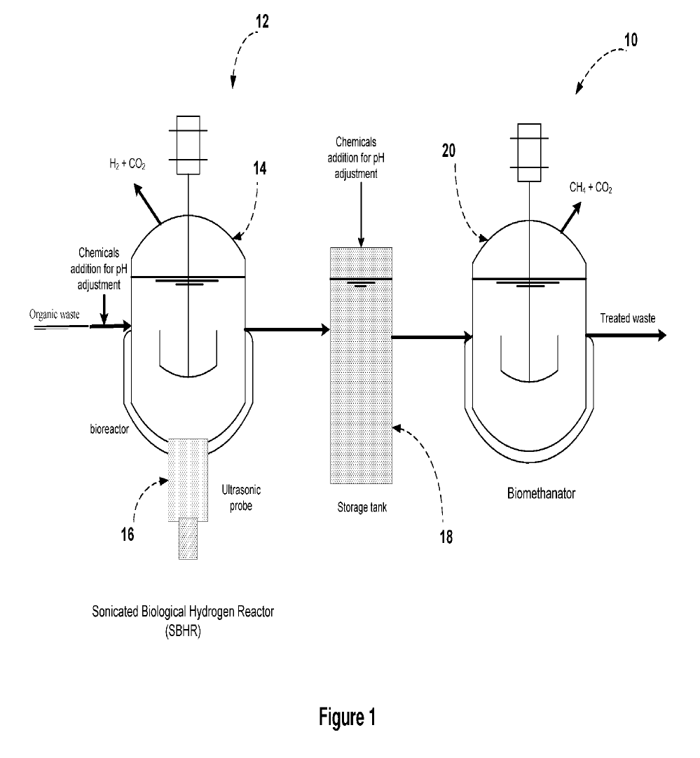

Referring to FIG. 1, an apparatus 10 for producing hydrogen and methane from organic waste is shown. Apparatus 10 comprises a sonicated biological hydrogen reactor (SBHR) 12 which includes a bioreactor 14 having an input for receiving organic waste into the bioreactor 14. A sonication energy source 16 is connected to the bioreactor. System 10 includes hydrogen producing microorganisms located in the bioreactor 14 which are utilized to break down the organic waste.

### Materials and Methods

### Systems Set Up and Operation

Two continuous-flow completely mixed bioreactors (10 cm diameter, 30 cm height) with a working volume of 2 L each were used. One bioreactor was a conventional continuous stirred tank reactor (CSTR) and the other, shown in FIG. 1, was sonicated biological hydrogen reactor (SBHR) 14. The two bioreactors each included a conventional continuous stirred tank reactor connected with a lab scale 2.5-inch diameter and the SBHR included an ultrasonic probe 16 at the bottom of the reactor (1 cm above the bottom of the reactor). The sonication pulses (inside the reactor) were set to 1 s on and 59 s off. The ultrasonic probe was supplied by Sonic and Materials (model VC-500, 500 W, and 20 kHz). These two systems (CSTR and SBHR) were operated on synthetic glucose-based feed for 90 days. The two reactors were seeded with 2 L of anaerobically digested sludge and maintained at a constant temperature of 37° C. After seeding, the two reactors were first operated in a batch mode for 24 h, after which the reactor was shifted to the continuous-flow mode with a hydraulic retention time (HRT) of 12 h. A summary of the operational conditions is shown in Table 1. The two systems were operated at two organic loading rates (OLRs): OLR-1 of 21.4 g COD/L·d with an influent glucose concentration of 10 g/L and OLR-2 of 32.1 g COD/L·d with an influent glucose concentration of 15 g/L.

TABLE 1
Operational conditions of the hydrogen production system

| | | Phase 1 | | Phase2 | |
|---|---|---|---|---|---|
| | Units | CSTR | SBHR | CSTR | SBHR |
| HRT | hours | 12 | 12 | 12 | 12 |
| Glucose concentration | 9/L | 10 | 10 | 15 | 15 |
| OLR | g COD/L | 21.4 | 21.4 | 32.1 | 32.1 |
| pH | | 5-6 | 5-6 | 5-6 | 5-6 |

### Inocula and Media Compositions

Anaerobic sludge was collected from the primary anaerobic digester at St Mary's wastewater treatment plant (St Mary's, Ontario, Canada) and used as seed sludge after sonication. The total suspended solids (TSS) and volatile suspended solids (VSS) concentrations of the sludge were 11 and 9 g/L, respectively. In order to enrich hydrogen-producing bacteria, the sludges were sonicated using a lab scale sonication device at specific energy of 20 kJ/g TS with temperature control as described in Elbeshbishy et al. [31]: the total sonication time was 20 minutes with the temperature not exceeding 30° C. and sonication alternating between 2 seconds on and 2 seconds off. The feed containing glucose at two different concentrations of 10 g/L (Phase 1) and 15 g/L (Phase 2), was supplied by 5 mL/L of a nutrient stock solution with the following composition per liter of stock: 1000 g NaHCO₃, 280 g NH₄Cl, 250 g of K₂HPO₄, 100 g of MgSO₄.7H₂O, 10 g of CaCl₂.2H₂O, 2 g of FeCl₂0.4H₂O, 0.05 g of H₃BO₃, 0.05 g of ZnCl₂, 0.03 g of CuCl₂, 0.5 g of MnCl₂.4H₂O, 0.05 g of (NH₄)₆Mo₇O₂₄, 0.05 g of AlCl₃, 0.05 g of CoCl₂0.6H₂O, and 0.05 g of NiCl₂.

### Analytical Methods

Biogas production was collected by wet tip gas meters (Gas Meters for Laboratories, Nashville, Tenn.). The gas meter consists of a volumetric cell for gas-liquid displacement, a sensor device for liquid level detection, and an electronic control circuit for data processing and display. Biogas composition including hydrogen, methane, and nitrogen was determined by a gas chromatograph (Model 310, SRI Instruments, Torrance, Calif.) equipped with a thermal conductivity detector (TCD) and a molecular sieve column (Molesieve 5A, mesh 80/100, 6 ft×⅛ in). The temperatures of the column and the TCD detector were 90 and 105° C., respectively. Argon was used as the carrier gas at a flow rate of 30 mL/min. The concentrations of volatile fatty acids (VFAs) were analyzed after filtering the sample through 0.45 mm filter paper using a gas chromatograph (Varian 8500, Varian Inc., Toronto, Canada) with a flame ionization detector (FID) equipped with a fused silica column (30 m×0.32 mm). Helium was used as the carrier gas at a flow rate of 5 mL/min. The temperatures of the column and detector were 110 and 250° C., respectively. TSS and VSS concentrations were analyzed using standard methods [32] and total and soluble chemical oxygen demand (TCOD, SCOD) was measured using HACH methods and test kits (HACH Odyssey DR/2500). Soluble parameters were determined after filtering the samples through 0.45 mm filter paper. Glucose was analyzed by anthrone-sulfuric acid method [24].

### Microbial Community Analysis

Under all four reactor conditions, at the end of each phase, the total genomic community DNA was extracted using the UltraClean Soil DNA Isolation Kit (MO BIO Laboratories, Carlsbad, Calif., USA) and after PCR amplification were analyzed by denaturing gradient gel electrophoresis (DGGE). The primer set of 357FGC (50-CGCCCGCCGCGCGCGGCGGGCGGGGCGGGGGCACGGGGGGCCTACGGGAGGCA GCAG-30) and 518R (50-ATTACCGCGGCTGCT GG-30) at the annealing temperature of 53° C. was used for PCR amplification of the variable V3 region of 16S rDNA from the purified genomic DNA. Denaturing gradient gel electrophoresis (DGGE) of PCR products was performed with a DCode universal mutation system (BioRad Laboratories, Hercules, Calif., USA). The PCR products were applied directly to 8% (w/v) polyacrylamide gel with 15-55% denaturant gradients. Electrophoresis was performed at a constant voltage of 130 V at 58° C. for 5 h. The DNA templates of the bands of interest were reamplified and the PCR products were purified using QIAquick PCR Purification Kit (Qiagen Sciences, MD, USA) in accordance with the manufacturer's protocol. The sequences of the reamplified DNA fragments were determined by dideoxy chain termination (Sequencing Facility, John P. Robarts Research Institute, London, Ontario) and compared with available sequences in the GenBank database using the BLAST program [25].

### Results

### Hydrogen Production

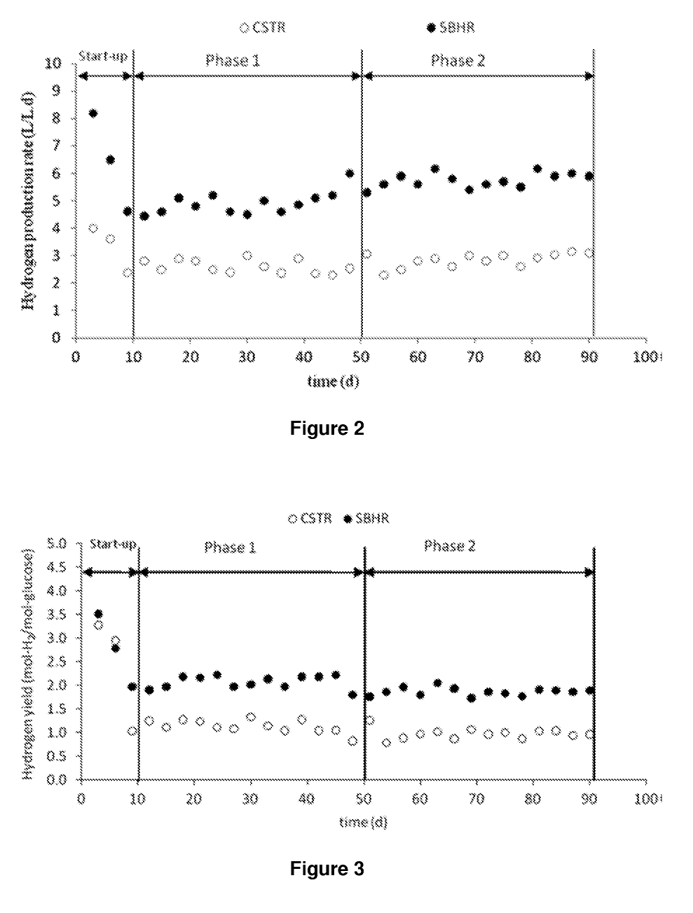

FIG. 2 illustrates the hydrogen production rates for the conventional CSTR and the SBHR at the two different OLRs of 21.4 (Phase 1) and 32.1 g COD/L·d (Phase 2). As apparent from FIG. 2, after the 10-day start up period, stable hydrogen production rates were observed in both the conventional CSTR and SBHR. The hydrogen production rates in the SBHR were significantly higher than those in the conventional CSTR at both OLRs. The average hydrogen production rates per unit reactor volume for the conventional CSTR were 2.6 and 2.8 L/L·d, as compared with 4.8 and 5.6 L/L·d for SBHR, in Phases 1 and 2, respectively. FIG. 3 shows the hydrogen yields for the conventional CSTR and the SBHR in the two phases. As depicted in FIG. 3, hydrogen yields of 1.2 and 1.0 mol H₂/mol glucose converted were observed for the CSTR in Phases 1 and 2, respectively, while for the SBHR, the hydrogen yields in Phases 1 and 2 were 2.1 and 1.9 mol H₂/mol glucose, respectively.

TABLE 2
Summary of steady-stated data in the hydrogen production systems

| Measured | | Phase 1 | | Phase 2 | |
|---|---|---|---|---|---|
| parameter | Units | CSTR | SBHR | CSTR | SBHR |
| Hydrogen | (L/L · d) | 2.6 ± 0.25 | 4.8 ± 0.3 | 2.8 ± 0.38 | 5.6 ± 0.51 |
| production rate | | | | | |
| Percentage | % | 38 ± 6 | 42 ± 3 | 35 ± 5 | 45 ± 2 |
| hydrogen | | | | | |
| Hydrogen yield | Mol H₂/mol | 1.2 ± 0.15 | 2.1 ± 0.23 | 1.0 ± 0.13 | 1.9 ± 0.21 |
| | glucose | | | | |
| Glucose | % | 92 ± 4 | 94 ± 2 | 76 ± 4 | 84 ± 4 |
| conversion | | | | | |
| Biomass | Mg/L | 1186 ± 69 | 1017 ± 81 | 1100 ± 64 | 939 ± 42 |
| concentration | | | | | |
| Biomass yield^{a} | (mg VSS/mg | 0.03 | 0.24 | 0.34 | 0.23 |
| | COD_{consumed}) | | | | |
| Specific H₂ | L/g VSS · d | 2.2 ± 0.3 | 4.7 ± 0.5 | 2.5 ± 0.3 | 6.2 ± 0.3 |
| production rate | | | | | |
| Acetate/butyrate | | 0.63 ± 0.19 | 1.13 ± 0.12 | 0.75 ± 0.17 | 1.20 ± 0.16 |

* Values represent averages ± standard deviations based on 12 steady-state samples.
^{a}Calculated based on the slop of the cumulative biomass produced versus the cumulative SCOD consumed.

Table 2 summarizes the steady-state data for the two systems during the two phases. Generally in biological treatment systems, steady-state data are collected after a minimum of 3 turnovers of the mean solids retention time (SRT). In addition to the aforementioned criteria, steady-state in this case also entailed less than 10% variation in biogas quantity, and reactor water quality parameters listed in the Analytical Methods section. The stability of both systems is evident from the very low coefficient of variation (CV), calculated as the standard deviation divided by the average of the steady-state data based on 12 samples. Glucose conversion efficiencies of 92% and 94% were achieved in Phase 1 for the CSTR and SBHR, respectively. In Phase 2, glucose conversion efficiencies decreased to 76% and 84% in the CSTR and SBHR. The conversion efficiency of glucose to hydrogen (based on the theoretical yield of 4 mol H₂/mol glucose) for the CSTR and SBHR were 23% and 51% in Phase 1, and 25% and 46% in Phase 2, respectively. Based on the aforementioned glucose conversion efficiencies, it is evident that by increasing the OLR, the glucose conversion decreased in the two systems, but in both phases, glucose conversion efficiencies in the SBHR were higher than that in the CSTR.

As shown in Table 2, the average hydrogen concentrations in the headspace of the conventional CSTR were 38% and 35% for the Phases 1 and 2, respectively, as compared with 42% and 46% in the SBHR, respectively.

### Volatile Fatty Acids (VFAs)

Hydrogen yield depends on the fermentation pathway and end-products [7 now 6]. The available hydrogen production from glucose is determined by the butyrate/acetate ratio [26]. When acetic acid is the end-product, there is a theoretical maximum of 4 mol hydrogen per mole glucose:

C₆H₁₂O₆+2H₂O→4H₂+2CH₃COOH+2CO₂ (1)

When butyrate is the end-product, there is a theoretical maximum of 2 mol hydrogen per mole glucose:

C₆H₁₂O₆+2H₂O→2H₂+CH₃CH₂CH₂COOH+2CO₂ (2)

The major VFAs detected were acetate (HAc), butyrate (HBu) and propionate (HPr). The HAc/HBu ratio has been examined in this study. As shown in Table 2, the HAc/HBu ratio in the SBHR was higher than in the CSTR in Phases 1 and 2. During Phase 1, HAc/HBu ratios of 0.63 and 1.13 were observed for the conventional CSTR and the SBHR, respectively, increasing to 0.75 and 1.20 in Phase 2 in both systems, respectively. The relationship between hydrogen yield and the corresponding values of HAc/HBu ratio for the two systems (data not shown) during the two phases shows that the hydrogen yield increased linearly with the increase in HAc/HBu ratio, consistent with past reports [27]. As shown in Table 3, the VFAs in the CSTR were higher than in the SBHR in both phases. The VFAs accounted for 92% of the effluent soluble COD for both CSTR and SBHR in Phase 1, as compared to 71% and 67% in the CSTR and SBHR in phase 2, respectively. Using the stoichiometric yields of 4 and 2 mol H₂/mol glucose from Eqs. (1) and (2), and according to the measured average concentrations of acetate and butyrate, the contribution of the two pathways was estimated. For the CSTR, the steady-state acetate concentrations ranged from 8154 mg/L to 10221 mg/L while the butyrate varied from 17308 mg/L to 20163 mg/L, with acetate and butyrate pathways contributing 41% and 59% of the hydrogen produced in Phase 1, and 43% and 57% in Phase 2, respectively. In the SBHR, the steady-state acetate concentrations ranged from 9317 mg/L to 12426 mg/L while the butyrate varied from 12360 mg/L to 15101 mg/L, with acetate and butyrate pathways contributing 53%, 47% of the hydrogen production in Phase 1 and 55%, 45% in Phase 2, respectively.

TABLE 3
Summary of products and COD mass balance

| Measured | | Phase 1 | | Phase 2 | |
|---|---|---|---|---|---|
| Parameter | Units | CSTR | SBHR | CSTR | SBHR |
| VSSₒᵤₜ | (mg COD/d)^{a} | 6739 ± 389 | 5775 ± 460 | 6248 ± 362 | 5335 ± 236 |
| SCODₒᵤₜ | (mg COD/d) | 28791 ± 1154 | 25420 ± 1097 | 49063 ± 1149 | 48520 ± 2100 |
| Glucoseₒᵤₜ | (mg COD/d)^{b} | 3833 ± 467 | 2833 ± 392 | 14490 ± 2572 | 10251 ± 1883 |
| Acetic acid | (mg COD/d) | 8154 ± 1234 | 9317 ± 748 | 10221 ± 823 | 12426 ± 1798 |
| Propionic | (mg COD/L) | 811 ± 46 | 898 ± 105 | 3111 ± 193 | 2956 ± 152 |
| Isobutyric | (mg COD/d) | 42 ± 12 | 106 ± 19 | 337 ± 39 | 397 ± 34 |
| Butyric | (mg COD/d) | 17308 ± 929 | 12360 ± 1140 | 20163 ± 1725 | 15101 ± 2097 |
| Isovaleric | (mg COD/d) | 17 ± 6 | 355 ± 76 | 496 ± 51 | 559 ± 48 |
| Valeric | (mg COD/d) | 104 ± 18 | 242 ± 37 | 556 ± 82 | 824 ± 71 |
| VFAs | (mg COD/d) | 26436 ± 1771 | 23279 ± 1664 | 34885 ± 1926 | 32263 ± 3158 |
| Ethanol | (mg COD/d) | 259 ± 33 | 339 ± 56 | 2297 ±313 | 2920 ± 86 |
| Hydrogen gas | (L/d) | 5.2 ± 0.5 | 9.6 ± 0.6 | 5.6 ±0.6 | 11.2 ± 1 |
| Hydrogen gas | (mg COD/d)^{c} | 3744 ± 360 | 6912 ± 432 | 4032 ± 432 | 8064 ± 720 |
| COD balance | (%)^{d} | 92 ± 3 | 89 ± 4 | 92 ±5 | 96 ± 7 |

* Values represent averages ± standard deviations based on 12 steady-state samples.
^{a}Based on 1.42 g COD/g VSS.
^{b}Based on 1.07 g COD/g Glucose.
^{c}Based on 8 g COD/g H2.
^{d}COD balance (%) = (VSSₒᵤₜ (g COD/d) + H₂ (g COD/d) + SCODₒᵤₜ (g COD/d))/(TCODᵢₙ (g COD/d)).

### Biomass Yield

The initial biomass concentration in the two reactors was 9 g VSS/L and it decreased sharply during the start up period (first 10 days). After the start up period, the biomass concentration in both the conventional CSTR and SBHR stabilized at average concentrations of 1.2 and 1.0 g VSS/L, respectively, during Phase 1. In Phase 2, as shown in Table 2, the biomass concentration in the two systems did not change significantly from Phase 1 (1.1 and 0.9 g VSS/L for the conventional CSTR and SBHR, respectively).

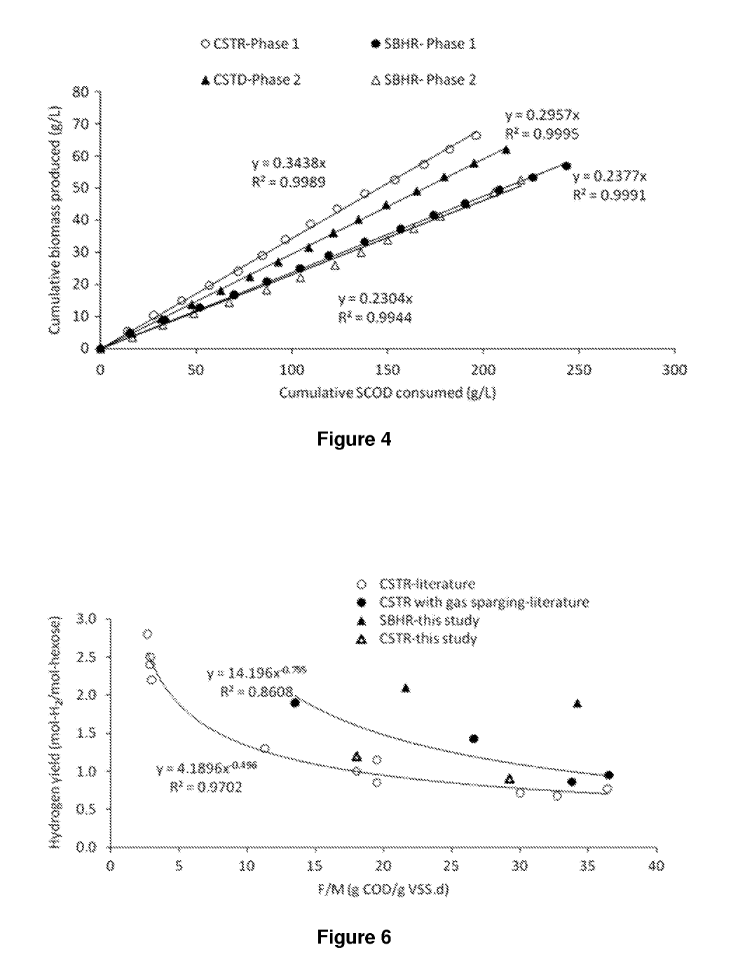

The biomass yield (as g VSS/g SCOD) was calculated based on the slope of the cumulative biomass produced versus the cumulative SCOD consumed (FIG. 4). As shown in FIG. 4, for the CSTR, the biomass yield increased from 0.30 to 0.34 g VSS/g SCOD when the OLR increased from 21.4 g COD/L·d to 32.1 g COD/L·d. The biomass yield of the SBHR remained constant at about 0.23 g VSS/g SCOD throughout the two phases. The biomass-specific hydrogen production rates were 2.2 and 2.5 L/g VSS·d in the CSTR in Phases 1 and 2, respectively, while in the SBHR, the specific hydrogen production rates were 4.7 and 6.2 Ug VSS·d in Phases 1 and 2, respectively.

The COD mass balances for the two systems in the two phases, calculated considering the measured influent and effluent CODs, and the equivalent CODs for both gas and biomass are shown in Table 3. The summation of COD balances of 89%-96% is an indication of the reliability of the data.

### Microbial Community Analysis

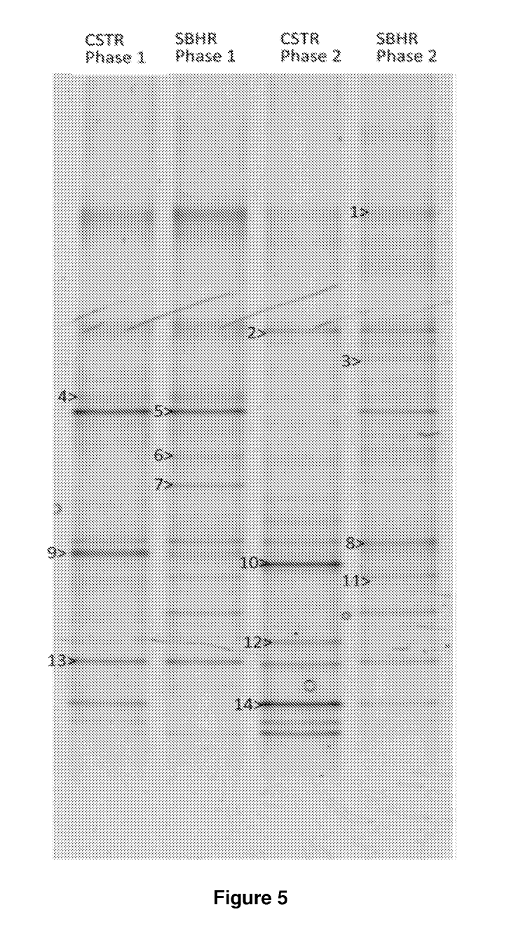

The microbial community structure was evaluated by extraction of total DNA from samples taken from the CSTR and SBHR, followed by PCR-DGGE. The DGGE profiles of the 16S rDNA gene fragment at each treatment condition are illustrated in FIG. 5. Table 4 shows the results of the sequence affiliation. In total, 14 bands and 11 species were identified. The number of the bands detected in SBHR (9 and 10 bands in Phases 1, and 2 respectively) was greater than the number detected in the CSTR (7 bands in each phase), indicating that ultrasonication increases microbial diversity. By excluding the uncultured bacterium, 6 and 5 species were identified for the CSTR in Phases 1 and 2, respectively, compared to 8 and 7 species for the SBHR.

TABLE 4
Affiliation of denaturation gradient gel electrophoresis (DGGE) fragments
determined by their 16S rDNA sequence

| | Affiliation | Similarity | Phase 1 | | Phase 2 | |
|---|---|---|---|---|---|---|
| Band | (Accession No. | (%) | CSTR | SBHR | CSTR | SBHR |
| 1 | Lactococcus sp. (EU689105.1) | 99 | x | x | x | x |
| 2 | Leuconostoc pseudomesenteroides | 96 | | | x | x |
| | (AB494729.1) | | | | | |
| 3 | Uncultured bacterium (FJ982841) | 95 | | | | x |
| 4 | Bacillus circulans (GQ478244.1) | 95 | x | x | | |
| 5 | Streptococcus gallolyticus (FN597254.1) | 100 | x | x | | x |
| 6 | Clostridium sp. (DQ986224.1) | 99 | | x | | x |
| 7 | Uncultured bacterium (FJ370100.1) | 100 | | x | | x |
| 8 | Clostridium butyricum (DQ831124.1) | 98 | x | x | x | x |
| 9 | Enterobacter cloacae (FP929040.1) | 100 | x | x | | |
| 10 | Clostridium acetobutyricum (FM994940.1) | 100 | | | x | |
| 11 | Citrobacter freundii (AB548829.1) | 100 | | x | | x |
| 12 | Uncultured bacterium (EF515734.1) | 98 | | | x | |
| 13 | Clostridium butyricum (AY458857.1) | 97 | x | x | x | x |
| 14 | Uncultured bacterium (EF515734.1) | 97 | x | | x | x |

Lactococcus sp. (band 1), Clostridium butyricum (band 7), and C. butyricum (band 13) were detected in both reactors in Phases 1 and 2. C. butyricum species is one of the most frequently reported species in hydrogen-producing mixed cultures [28,29]. Lactococcus sp. (band 1) observed in the two bioreactors in the two phases is known as a lactic acid producing bacterium [30]. Bacillus circulans (band 4) and Enterobacter cloacae (band 9) were detected in both systems in Phase 1 only, while Leuconostoc pseudomesenteroides (band 2) was detected in Phase 2 only. Clostridium acetobutyricum (band 10) was detected in the CSTR in Phase 2 only. C. acetobutyricum ferments carbohydrates to hydrogen and carbon dioxide with acetate and butyrate as the main soluble metabolites [31]. E. cloacae has been reported as one of the dominant populations in hydrogen-producing biomass with molasses wastewater from a sugarbeet or glucose refinery as a substrate [32]. Oxidation reduction potential (ORP) decreased rapidly in the presence of B. circulans, and an anaerobic environment suitable for the growth of anaerobic and hydrogen-producing bacteria was established [33]. Clostridium sp. (band 6) and Citrobacter freundii (band 11) were detected in the SBHR and not detected in the CSTR either in Phase 1 or Phase 2. The diversity of the species appears to have a positive effect on biohydrogen production while ultrasonication apparently did not affect the lactic acid producing bacteria.

The hydrogen production rate of SBHR with respect to CSTR has thus been shown to increase 85% and 100% in Phases 1 and 2, respectively. Similarly, the percentage increases in the hydrogen yield were 75% and 90% in Phases 1 and 2, respectively. For both the CSTR and the SBHR, the hydrogen production rate increased with increasing OLR, while the hydrogen yield decreased with increasing the OLR from 21.4 to 32.1 g COD/L·d. The decrease in hydrogen yield with the increase of OLR may be due to incomplete conversion of glucose. The hydrogen content in the SBHR headspace was higher than that in the CSTR by 10% and 31% in Phases 1 and 2, respectively. As evident from the aforementioned values, the hydrogen content in the headspace did exhibit a significant improvement, which may be attributable to ultrasonication hastening the exit of dissolved CO₂ and H₂ from the liquid. Kim et al. [34] achieved a maximum hydrogen yield of 1.68 mol H₂/mol hexose consumed using CO₂ sparging at flow rate of 60 mL/min·L_{reactor}, with a 118% increase compared with the control reactor at 0.77 mol H₂/mol hexose consumed, but observed only a 25% increase in hydrogen yield using N₂ sparging at the same flow rate. In another study, Kraemer et al. [35] reported that the hydrogen yield increased from 1.0 to 2.0 mol H₂/mol glucose with N₂ sparging at flow rate of 12 mL/min·L_{reactor}. The use of ultrasonication to enhance the hydrogen production thus achieved higher hydrogen yields compared with these.

FIG. 6 shows the relationship between the food to microorganisms (F/M) ratio and the hydrogen yield using the results obtained by the inventors and seven literature studies, three of which used gas sparging to enhance the hydrogen production from a CSTR [7, 36, 28] and the others for conventional CSTR [37,38,11]. As shown in FIG. 6, for the CSTR systems (two in this study and seven from the literature), at an F/M below 5 g COD/g VSS·d, the hydrogen yield decreased sharply with increasing the F/M ratio, while after that a smooth decline in the hydrogen yield is observed upon increasing the F/M. The hydrogen yield in the CSTR for F/M ratios higher than 20 g COD/g VSS·d seems to be constant at an average value of about 0.8 mol H₂/mol hexose, while for CSTRs with gas sparging, the hydrogen yields are higher than in the CSTR. As depicted in the FIG. 6, it is evident that the effect of gas sparging in the enhancement of hydrogen yield is significant (about 60% increase) at F/M ratios below 26 g COD/g VSS·d, while at F/M ratios above 26 g COD/g VSS·d, the enhancement in hydrogen production is not significant at about 20%. Although the hydrogen yields of the two CSTR systems described here (hollow triangles) match literature values as shown in the FIG. 6, the hydrogen yields of the SBHR (solid triangle) are higher than both the CSTR alone and CSTR with gas sparging even at high F/M ratio. The data presented in FIG. 6 highlights the beneficial impact of ultrasonication inside the reactor at all ranges of F/M ratios. The hydrogen yield from the SBHR is higher than that of the CSTRs with gas sparging by about 40% and 60% at OLR of 24.1 and 32.1 g COD/L·d, respectively.

As shown in Table 3, the acetic acid in the SBHR was generally higher than in the CSTR in both phases, in contrast with the butyric acid which was higher in the CSTR. The contribution of the acetate pathway to hydrogen production in the SBHR was on average 28% higher than in the CSTR. The propionic acid concentrations in both reactors were comparable in both phases, although the propionic acid increased sharply in Phase 2 in both reactors. The same trend has been observed for ethanol concentration; it was very low in Phase 1 and increased sharply in Phase 2, which may be due to the microbial shift as indicated by the DGGE analysis (Table 4). L. pseudomesenteroides, which is known as a lactic acid producer [30] was observed in Phase 2 only. This microbial shift might explain the decrease in hydrogen production rate, hydrogen yield, and glucose conversion in Phase 2 compared with Phase 1. On the other hand, as Clostridium is a widely reported species in high hydrogen production systems and C. freundii is also a hydrogen-producing bacteria [39], the DGGE results substantiate that the observed higher hydrogen yield in the SBHR compared with the CSTR may be due to the microbial shift as two different hydrogen producers (Clostridium sp. and C. freundii), were detected in the SBHR and not in the CSTR.

The biomass yield in the SBHR was lower than that of conventional CSTR by 18% and 32% in Phases 1 and 2, respectively. The observed inverse relationship between the biomass yield and hydrogen yields is consistent with earlier findings of Hafez et al. [40] who observed similar trends, using data from their CSTR and literature studies.

The mechanisms for enhancement of hydrogen production obtained through the use of ultrasonication may be due to one or more of the following: (1) decreasing the dissolved hydrogen concentration, (2) enhancement of the mass transfer, (3) increasing the microorganisms' growth rate and/or (4) solubilization. Decreasing the dissolved H₂ concentration is known to increase the H₂ production via one of two possible scenarios: (i) increase the H₂ production, or (ii) decrease the H₂ consumption. H₂ generation is mediated by hydrogenase using electrons from ferreodoxin (Fd) to reduce protons. On the other hand, higher H₂ yields during N₂ sparging may be caused by decreased H₂ consumption. H₂ consumption may be via homoacetogenesis or methanogenesis and as in most cases there was no reported detection of methane production in the hydrogen production reactors due to the high dilution rate and the low pH. Therefore, the main mechanism responsible for the consumption of H₂ is the homoacetogenesis, which reduces dissolved CO₂ using the dissolved H₂ to produce acetate [41]. Mizuno et al. [11] and Kim et al. [28] reported that the increase in H₂ production using gas sparging is due to the decrease of dissolved H₂ concentration and hence enhancement of the activity of the relevant H₂-producing enzymes. Kraemer and Bagley [35] who observed an increase in H₂ production at a dissolved H₂ concentration of 485 mM, much greater than the threshold concentration of 0.5 m M below which H₂ production increased, attributed the increase to a decrease in the rate of dissolved H₂ consumption.

Ultrasound has been reported to enhance some multiphase chemical reactions, by affecting the yield of the reaction and/or its selectivity [42]. Chisti [14] attributed part of the beneficial effects of ultrasound in biotechnology to mass transfer improvements, not only increased mass transfer around the cells (improving the exchanges of nutrients and products), but also inside the cells [43,44]. Kumar et al. [45] investigated gas-liquid mass transfer with a 20 kHz ultrasonic horn, and concluded that low frequency (20 kHz) appeared more favourable than high frequency (500 kHz). The aforementioned researchers attributed the observed enhancement of mass transfer to a reduction in gas bubble size. Moreover, intermittent-power low-frequency ultrasound of short duration can enhance a productivity of live microbial systems [14]. It was found that low-frequency ultrasound (70 kHz) of low acoustic intensity (<2 W/cm²) increased the growth rate of cells compared to growth without ultrasound [46]. Guo et al. [18] who reported an increase in hydrogen production when they applied ultrasonication on the substrate and/or on the seed, attributed the increase to the solubilization and increase of SCOD. The specific ultrasonication energy required for cell lysis is not widely reported in the literature, and is primarily derived from the solubilization of cell protein data.

Elbeshbishy et al. [47] reported that a minimum specific ultrasonication energy of 500 kJ/kg TS is required for initiation of cell protein solubilization from hog manure while Wang et al. [48] reported that cell protein solubilization from WAS was maximum at a specific energy of 7700 kJ/kg TS. A significant variability in ultrasonication energy requirement for cell lysis is observed due to biomass nature, source, and characteristics. Previous work by the inventors named herein on batch systems [22] indicated that ultrasonication energy of 20000 kJ/kg TS inhibited methanogenic bacteria and did not adversely impact biohydrogen producers.

As mentioned previously, the apparatus used to obtain the results described herein is shown in FIG. 1. In addition to the bioreactor 14, used for hydrogen production, system 10 includes a biomethanator 20 which may have a sonication energy source connected to the biomethanator 20 located downstream of the SBHR 12 and hydraulically connected with an output of the bioreactor 14. In operation, the organic waste (labelled organic waste in FIG. 1) entering the sonicated biological hydrogen production 12 is sonically disrupted by the sonication energy source 16 and broken down microbiologically by hydrogen producing microorganisms to predominantly hydrogen gas and carbon dioxide, and a mixture of volatile fatty acids and primary alcohols in the bioreactor 14. The hydrogen gas and carbon dioxide are emitted from the bioreactor 14, and a SBHR effluent flows into the biomethanator 20 wherein the residual organics are broken down microbiologically predominantly to methane gas and carbon dioxide. The methane gas and carbon dioxide produced in the biomethanator 20 are emitted and liquid waste (labelled treated waste in FIG. 1) containing residual organics is discharged from the biomethanator 20.

Optionally, apparatus 10 includes a storage tank 18 hydraulically connected to the SBHR 12 located downstream of the SBHR 12 and which is located upstream of the biomethanator 20 and hydraulically connected to both the SBHR 12 and biomethanator 20 for adjusting loading rates of the liquids and the pH entering the biomethanator 20, as appropriate e.g., depending upon the HRT of the biomethanator.

The apparatus may include dispenser refer to for dispensing chemicals into the storage tank 18 for adjusting alkalinity and pH of the liquid in the storage tank 18.

The apparatus also preferably includes temperature controllers for controlling the temperature in the SBHR 12 and in the biomethanator 20. A typical temperature range in which the temperature of the contents of both SBHR 12 and biomethanator 20 is maintained is between from about 20° C. to about 85° C.

The apparatus may also include dispenser refer to for dispensing nutrients and pH adjustment compounds into the SBHR 12 and biomethanator 20. The nutrients may be, but are not limited to, any one or combination of nitrogen containing compounds, phosphorous containing compounds, and trace metals including iron, manganese, magnesium, calcium, cobalt, zinc, nickel, and copper. The pH adjustment compounds include, but are not limited to soda ash, sodium bicarbonate, sodium hydroxide, calcium hydroxide, magnesium hydroxide, nitric acid, and hydrochloric acid.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

The disclosures of all references cited herein are incorporated herein in their entirety, as though they had been reproduced herein.

The foregoing description of exemplary and preferred embodiments of the invention has been presented to illustrate the principles of the invention and not to limit the invention to the particular embodiment illustrated. It is intended that the scope of the invention be defined by all of the embodiments encompassed within the following claims and their equivalents.

### REFERENCES

[1] M. T. Skonieczny, Biological hydrogen production from industrial wastewater with Clostridium beijerinckii, Master of Engineering, Department of Chemical Engineering, McGill University, Montreal, 2008.
[2] F. R. Hawkes, I. Hussy, G. Kyazze, R. Dinsdale, D. L. Hawkes, Continuous dark fermentative hydrogen production by mesophilic microflora: principles and progress, Int J Hydrogen Energy 32 (2007), 172-184.
[3] N. Kataoka, A. Miya, K. Kiriyama, Studies on hydrogen production by continuous culture system of hydrogen-producing anaerobic bacteria, Water Sci Technol 36 (1997), 41-47.
[4] P. A. M. Claassen, J. B. van Lier, A. M. Lopez Contreras, E. W. J. van Niel, L. Sijtsma, A. J. M. Stams, S. S. de Vries, R. A. Weusthuis, Utilisation of biomass for the supply of energy carriers, Appl Microbiol Biotechnol 52 (1999), 741-755.
[5] W. M. Alalayah, M. S. Kalil, A. A. H. Kadhum, J. Md. Jahim, N. M. Alauj, Effect of Environmental Parameters on hydrogen Production using Clostridium Saccharoperbutylacetonicum N1-4(ATCC 13564), Am J Environ Sci 5 (2009), 80-86.
[6] D. B. Levin, L. Pitt, M. Love, Biohydrogen production: prospects and limitations to practical application, Int J Hydrogen Energy 29 (2004), 173-85.
[7] O. Mizuno, R. Dinsdale, F. R. Hawkes, D. L. Hawkes, T. Noike, Enhancement of hydrogen production from glucose by nitrogen gas sparging, Bioresource Technol 73 (2000), 59-65.
[8] J. T. Kraemer, D. M. Bagley, Improving the yield from fermentative hydrogen production, Biotechnol Lett 29 (2007), 685-695.
[9] R. J. Lamed, J. H. Lobos, T. M. Su, Effects of stirring and hydrogen on fermentation products of Clostridium thermocellum, Appl Environ Microbiol 54 (1988), 1216-1221.
[10] T. M. Liang, S. S. Cheng, K. L. Wu, Behavioral study on hydrogen fermentation reactor installed with silicone rubber membrane, Int J Hydrogen Energy 27 (2002), 1157-1165.
[11] S. Y. Wu, C. H. Hung, C. N. Lin, H. W. Chen, A. S. Lee, J. S. Chang, Fermentative hydrogen production and bacterial community structure in high-rate anaerobic bioreactors containing silicone-immobilized and self-flocculated sludge. Biotechnol Bioeng 93(2006), 934-946.
[12] A. Tiehm, K. Nickel, U. Nies, The use of ultrasound to accelerate the anaerobic digestion of sewage sludge, Water Sci Technol 36 (1997), 121-128.
[13] Y. Chisti, Sonobioreactors: using ultrasound for enhanced microbial productivity, Trends Biotechnol 21 (2003), 89-93.
[14] Y. Chisti, M. Moo-Young, Disruption of microbial cells for intracellular products, Enzyme Microb Technol 8 (1986), 194-204.
[15] S. G. Pavlostathis, J. M. Gossett, A kinetic model for anaerobic digestion of biological sludge, Biotechnol. Bioeng. 27 (1986) 1519-1530.
[16] S. I. Perez-Elvira, P. Nieto Diez, F. Fdz-Polanco, Sludge minimisation technologies, Rev. Environ. Sci. Biotechnol. 5 (2006) 375-398.
[17] Wang C C, Chang C W, Chu C P, Lee D J, Chang B V, Liao C S. Producing hydrogen from wastewater sludge by Clostridium bifermentans. J Biotechnol 2003a; 102:83-92.
[18] Guo L, Li X M, Bo X, Yang Q, Zeng G M, Liao D, et al. Impacts of sterilization, microwave and ultrasonication pretreatment on hydrogen producing using waste sludge. Bioresour Technol 2008; 99:3651-8.
[19] BenYi X, JunXin L. Effect of various pretreatments on biohydrogen production from sewage sludge. Chin Sci Bull 2009; 54:2038-44.
[20] Guo Y P, Kim S H, Sung S H, Lee P H. Effect of ultrasonic treatment of digestion sludge on bio-hydrogen production from sucrose by anaerobic fermentation. Int J Hydrogen Energy; 2010; doi:10.1016/j.ijhydene 0.2010.01.09.
[21] More T T, Ghangrekar M M. Improving performance of microbial fuel cell with ultrasonication pre-treatment of mixed anaerobic inoculum sludge. Bioresour Technol 2010; 101:562-7.
[22] Elbeshbishy E, Hafez H, Nakhla G. Enhancement of biohydrogen producing using ultrasonication. Int J Hydrogen Energy; 2010; doi:10.1016/j.ijhydene 0.2010.03.119.
[23] APHA, AWWA, WEF. Standard methods for examination of water and wastewater. 19th ed.; 1995.
[24] Dubois M, Gilles K A, Hamilton J K, Rebers P A, Smith F. Colorimetric method for determination of sugars and related substances. Anal Chem 1956; 28:350-6.
[25] Altschul S F, Gish W, Miller W, Myers E W, Lipman D J. Basic local alignment search tool. J Mol Biol 1990; 215: 403-10.
[26] Reith J, Wijffels R, Barteb H. Bio-methane & Bio-hydrogen: status and perspectives of biological methane and hydrogen production, http://gasunie.eldoc.ub.rug.nl/root/2003/3339875/.
[27] Wang J L, Wan W. The effect of substrate concentration on biohydrogen production by using kinetic models. Sci China B Chem Life Sci Earth Sci 2008; 51:1110-7.
[28] Kim D H, Han S K, Kim S H, Shin H S. Effect of gas sparging on continuous fermentative hydrogen production. Int J Hydrogen Energy 2006a; 31:2158-69.
[29] Wang J L, Wan W. Comparison of different pretreatment methods for enriching hydrogen-producing bacteria from digested sludge. Int J Hydrogen Energy 2008; 33: 2934-41.
[30] Ennahar S, Cai Y, Fujita Y. Phylogenetic diversity of lactic acid bacteria associated with paddy rice silage as determined by 16S ribosomal DNA analysis. Appl Environ Microbiol; 2003: 444e51.
[31] Woods D R. The clostridia and biotechnology, USA. Butterworth-Heinemann; 1993.
[32] Xing D, Ren N, Rittmann B E. Genetic diversity of hydrogen-producing bacteria in an Acidophilic Ethanol-H₂-Coproducing system, analyzed using the [Fe]-Hydrogenase gene. Appl Environ Microbiol 2008; 74:1232-9.
[33] Haruta S, Cui Z, Huang Z, Li M, Ishii M, Igarashi Y. Construction of a stable microbial community with high cellulose-degradation ability. Appl Microbiol Biotechnol 2002; 59:529-34.
[34] Liu D W, Liu D P, Zeng R J, Angelidaki I. Hydrogen and methane production from household solid waste in the two-stage fermentation process. Water Res 2006; 40:2230-6.
[35] Kraemer J T, Bagley D M. Supersaturation of dissolved H₂ and CO₂ during fermentative hydrogen production with N2 sparging. Biotechnol Lett 2006; 28:1485-91.
[36] Hussy I, Hawkes F R, Dinsdale R, Hawkes D L. Continuous fermentative hydrogen production from sucrose and sugarbeet. Int J Hydrogen Energy 2005; 30:471-83.
[37] Oh S E, Lyer P, Bruns M A, Logan B E. Biological hydrogen production using a membrane bioreactor. Biotechnol Bioeng 2004; 87:119-27.
[38] Van Ginkel S W, Logan B. Increased biological hydrogen production with reduced organic loading. Water Res 2005; 39: 3819-26.
[39] Kumar G R, Vatsala T M. Hydrogen production from glucose by Citrobacter freundii. Indian J Exp Biol 1989; 27: 824-5.
[40] Hafez H, Nakhla G, El. Naggar H M, Elbeshbishy E, Baghchehsaraee B. Effect of organic loading on a novel hydrogen bioreactor. Int J Hydrogen Energy 2010; 35:81-92.
[41] Wood H G, Ragsdale S W, Pezacka E. The acetyl-CoA pathway of autotrophic growth. FEMS Microbiol Rev 1986; 39(1986): 345-62.
[42] Mason T J, Critical report on applied chemistry. In: Mason T J, editor. Chemistry with ultrasound, vol. 28. Elsevier; 1990. Applied Science, published for the Society of Chemical Industry.
[43] BarR. Ultrasound enhanced bioprocesses: cholesterol oxidation by Rhodococcus erythropolis. Biotechnol Bioeng 1988; 32:655-63.
[44] Sinisterra J V. Application of ultrasound to biotechnology: an overview. Ultrasonics 1992; 30:180-5.
[45] Kumar A, Gogate P R, Pandit A B, Delmas H, Wilhelm A M. Investigating mass transfer efficacy of sonochemical reactors. Ind Eng Chem Res 2004; 43:1812-9.
[46] Pitt W G, Ross S A. Ultrasound increases the rate of bacterial cell growth. Biotechnol Prog 2003; 19:1038-44.
[47] Elbeshbishy E, et al. Impact of ultrasonication of hog manure on anaerobic digestability. Ultrason Sonochem; 2010; doi:10.1016/j.ultsonch.2010.04.011.
[48] Wang F, Lu S, JiM. Components of released liquid from ultrasonic waste activated sludge disintegration. Ultrason Sonochem 2006; 13:334-8.

1. A method of anaerobically digesting organic material, the method comprising the steps of:
(a) continuously fermenting a mixture of a hydrogen-producing anaerobic microorganism and a portion of the organic material in a sonicated biological hydrogen reactor to produce hydrogen, wherein said hydrogen reactor is a completely mixed bioreactor having a sonication probe located therein;
(b) drawing gaseous hydrogen produced in step (a) from a headspace above the mixture in the bioreactor;
(c) feeding another portion of the material having an organic load (OL) into the bioreactor to supplement the organic load of the material in the bioreactor; and
(d) removing a portion of fermented material from the bioreactor; wherein:
step (a) includes enriching the hydrogen-producing bacteria and inhibiting methanogenesis by intermittently sonicating the mixture up to 90% of the entire time during which the mixture is in the completely mixed bioreactor and step (c) is repeated or performed continuously and step (d) is repeated or performed continuously, to obtain a hydraulic residence time (HRT) of the organic material in the bioreactor of between 2 and 48 hours and an average rate of production of hydrogen in step (a) that is at least 20% of the average organic loading rate (OLR) when the rate of hydrogen production is measured in liters hydrogen per liter bioreactor volume per unit time (L/L_{bioreactor}·d) and the OLR is measured as g COD per liter bioreactor volume per unit time (g COD/L_{bioreactor}·d).

2. The method of claim 1 wherein the microorganism is selected from the group of bacteria, archaea, protozoa, and fungi.

3. The method of claim 1, wherein the fermentation mixture comprises at least one strain of Acetobacter sp., Gluconobacter sp., or Clostridium sp.

4. The method of claim 1, wherein the microorganism comprises mesophilic bacteria, and the method further includes maintaining the temperature of the bioreactor between 30° C. and 45° C.

5. The method of claim 1, wherein the microorganism comprises thermophilic bacteria, and the method further includes maintaining the temperature of the bioreactor between 50° C. and 65° C.

6. The method of claim 1, further comprising maintaining the pH of the mixture in the bioreactor between 4.5 and 6.5.

7. The method of claim 6, wherein the pH is maintained between 5 and 6.

8. The method of claim 1, wherein the HRT is between 2 hours and 24 hours.

9. The method of claim 3, wherein the HRT is between 2 hours and 18 hours.

10. The method of claim 6, wherein the HRT is between 4 and 6 hours.

11. The method of claim 1, including intermittently sonicating the mixture from 1% of the time to 80% of the time.

12. The method of claim 4, including intermittently sonicating the mixture between 1% of the time and 70% of the time.

13. The method of claim 5, including intermittently sonicating the mixture between 5% and 60% of the time.

14. The method of claim 1, further comprising the step of agitating the mixture in the bioreactor.

15. The method of claim 14, wherein agitating the mixture includes mechanically agitating the bioreactor.

16. The method of claim 1, wherein step of intermittently sonicating includes applying a sonication frequency to the mixture with the same frequency maintained throughout all sonicating step.

17. The method of claim 16, wherein the sonication frequency is in the range of 1 to 500 kHz.

18. The method of claim 17, wherein the sonication frequency is in the range of 20 to 500 kHz.

19. The method of claim 1, wherein the organic material comprises low solids content wastewaters and soluble feedstocks.

20. The method of claim 19, wherein the organic material comprises an alcohol, a ketone, an aldehyde, a volatile fatty acid, an ester, an ether, or a combination of any of the preceding.

21. The method of claim 1, wherein the organic material comprises one or any combination of a polysaccharide and a monosaccharide.

22. (canceled)

23. (canceled)

24. The method of claim 2, wherein the bioreactor further comprises a temperature controller.

25. The method of claim 1, further comprising adding one or more nutrients to the bioreactor, the nutrient(s) being one or more of nitrogen containing compounds, phosphorous containing compounds, iron, manganese, magnesium, calcium, cobalt, zinc, nickel, copper.

26. The method of claim 1, wherein steps (a) to (d) are performed for a period of at least three days.

27. The method of claim 1, wherein hydrogen-producing anaerobic microorganisms are supplied into the bioreactor as a component of sludge at least once.

28. The method of claim 1, wherein said intermittently sonicating comprises powering the probe on and off.

29. The method of claim 28, wherein the probe is in direct contact with the mixture.

30. The method of claim 1, wherein in the vessel comprises an outlet through which said portion of fermented material is removed from the bioreactor.

31. The method of claim 30, wherein the outlet is connected to a conduit connected to a biomethanator downstream of the bioreactor, for delivery of said the fermented material to the biomethanator.

32. The method of claim 31, wherein products of the fermentation include carbon dioxide, volatile fatty acids and alcohols and further comprising delivering said products to the biomethanator.

33. The method of claim 31, wherein the conduit comprises an in-line chamber located intermediate the bioreactor and the biomethanator, and the method further comprises delivering a said portion of fermented material from the bioreactor to the chamber, and adjusting the pH of the material therein.


SEQUENCE LISTING (2 sequences)

SEQ ID NO: 1 - DNA, 57 bp, Artificial Sequence
  Primer
cgcccgccgcgcgcggcgggcggggcgggggcacggggggcctacgggaggcagcag

SEQ ID NO: 2 - DNA, 17 bp, Artificial Sequence
  Primer
attaccgcggctgctgg